(19) United States
(12) Patent Application Publication
(10) Pub. No.: US 2009/0234386 A1
(43) Pub. Date: Sep. 17, 2009

(54) Suture Cleat for Soft Tissue Injury Repair

(76) Inventors: John C. Dean, Midland, TX (US); Jonathan H. Webb, Midland, TX (US)

Correspondence Address: WONG, CABELLO, LUTSCH, RUTHERFORD & BRUCCULERI,;L.L.P., 20333 SH 249 6th Floor, HOUSTON, TX, 77070, US

(21) Appl. No.: 12/045,832

(22) Filed: Mar. 11, 2008

(51) Int. Cl. A61B 17/04

(52) U.S. Cl. 606/232

(57) ABSTRACT

Various suture cleats are disclosed for repairing soft tissue injuries. The cleats have a body and one or more spikes extending from one side and embedding into the soft tissue. A suture has a distal end attached to one of the suture cleats and passes through passages in one or more other suture cleats in repairing the soft tissue injury. The spikes are designed to embed only partially into the soft tissue. For example, two cleats can position on the top and bottom sides of the soft tissue on one side of the injury with a portion of suture interconnecting the two cleats though the tissue. In another example, one of the cleats can include a post that is positioned through the soft tissue and to which the suture connects. The distal end of the suture can fix to another suture cleat, a suture anchor, a bone tunnel, or a screw.

### BACKGROUND

Tearing or avulsion of soft tissue from bone is a relatively common type of injury, especially in sports, and can occur in many types of orthopedic injuries, such as torn or ruptured tendons and/or ligaments. In the shoulder, for example, portion of the rotator cuff tendons can tear within themselves or avulse from their insertion into the bone. FIGS. 1A-1B show superior views of a shoulder having a typical torn rotator cuff. Here, the tear is associated with the supraspinatus muscle as it inserts into the humerus. The subscapularis muscle and the coracoid process are also shown in FIG. 1A for reference.

The tear 10A shown in FIG. 1A is a simple tear and is generally perpendicular to the line of action of the muscle. In FIG. 1B, however, the tear 10B is more complex because the tear branches both parallel and normal to the muscle fibers. In either case, such a torn rotator cuff can lead to pain, weakness, and loss of function.

In many cases, the rotator cuff is repaired by surgically reconnecting the edges of the torn muscle or tendon. Repairs may also include reconnecting the edges of any interstitial tear in the tendons, as well as approximating or reattaching the torn edge of the soft tissue to the bone where it originated. Common techniques for repairing tears to soft tissue and the avulsion of soft tissue from bone include using sutures through bone tunnels, suture anchors, friction anchors, tacks, screws with spiked washers and staples, or any combination of these techniques.

Any repair of a rotator cuff injury should have a secure fixation to soft tissue and should preserve the range of motion through which a muscle is expected to function after the repair. The fixation should also serve to provide a means for the soft tissue to anatomically reattach to a position in the shoulder, the humeral head in this case. In the shoulder, the soft tissues may experience wide ranges of motion, as shown by the views in FIGS. 2A-2B of a shoulder during internal and external rotations. In addition to these rotations, the shoulder may also be moved through adduction and abduction motions (not shown). The various motions indicate that the soft tissue may undergo dramatic variations in stresses and that a wide variation in possible stresses at a particular point can occur. A surgical repair of injured soft tissue, such as the tears shown in FIGS. 1A-1B, preferably accounts for different requirements at various points along the injured site in order to alleviate concerns associated with the repair.

### BRIEF DESCRIPTION OF THE DRAWINGS

FIGS. 1A-1B are superior views of a shoulder and a rotator cuff demonstrating a tear in the rotator cuff.

FIGS. 2A-2B are superior views of the shoulder and rotator cuff in full internal rotation and full external rotation, respectively.

FIG. 3A is a top perspective view of a suture cleat according to one embodiment of the present disclosure.

FIG. 3B is a bottom perspective view of the suture cleat in FIG. 3A.

FIGS. 3C-3F illustrates techniques for attaching an end of a suture to the suture cleat of FIG. 3A.

FIG. 4A is side view showing two suture cleats of FIG. 3A used in soft tissue repairs.

FIG. 4B shows the side view of FIG. 4A when force is applied to the suture cleats.

FIG. 5A is a side view showing one suture cleat of FIG. 3A used in soft tissue repairs.

FIG. 5B shows the side view of FIG. 5A when force is applied to the suture cleat.

FIG. 6A is a perspective view of a suture cleat having a post according to yet another embodiment of the present disclosure.

FIG. 6B is a side view showing the suture cleat of FIG. 6A used in soft tissue repairs.

FIG. 6C shows the side view of FIG. 6B when force is applied to the suture cleat.

FIGS. 7A-7C show alternative embodiments of suture cleats having posts.

FIG. 8A is a superior view illustrating suture cleats to repair a rotator cuff tear.

FIG. 8B is a cross-sectional view illustrating suture cleats, sutures, and bone tunnels to repair a rotator cuff tear.

FIG. 8C is a cross-sectional view illustrating suture cleats, sutures, and a screw to repair a rotator cuff tear.

FIG. 8D is a cross-sectional view illustrating suture cleats, sutures, and a suture anchor to repair a rotator cuff tear.

FIG. 9 is a superior view of an injured shoulder having a torn rotator cuff repaired using interrupted sutures and augmented with an embodiment of the suture cleats.

FIG. 10 is a superior view of an injured shoulder having a torn rotator cuff repaired using interrupted sutures and augmented with an embodiment of the suture cleats.

### DETAILED DESCRIPTION

A suture cleat 100 according to one embodiment is illustrated in FIGS. 3A-3B. The suture cleat 100 has a disk body 102 with a first (top) side 104 and a second (bottom) side 106. The second side 106 is intended to position against soft tissue with a plurality of spikes 108 extending from the second side 106 embedding into the soft tissue. The body 102 also defines a passage 105 therethrough for suture. For the sake of illustration, the cleat's body 102 in one implementation may have a diameter of about 6-mm and may fit within a space of 8.5-mm to effectuate the desired soft tissue repairs of a torn rotator cuff. The length of the spikes 108 may vary depending on the implementation and intended use of the cleat 100.

Suture can attach to the cleat 100 using several techniques. In FIG. 3C, for example, a Mulberry knot or other large knot 55 can be made on the suture 50's end. Alternatively as shown in FIG. 3D, the end of the suture 50 can be tied to an independent anchor or cross member 56. Either way, the suture 50 can be passed through the suture passage 105 until this knot 55 or cross member 56 engages the passage 105 and is prevented from passing further. Alternatively as shown in FIG. 3E, a cross member 57 can be disposed in the cleat's suture passage 105 allowing the end of the suture 50 to tie thereto. In yet another alternative shown in FIG. 3F, the cleat 100 can be fabricated with the suture 50's end already embedded in the cleat's material when formed so that the suture 50 and cleat 100 are integrally connected. As further shown, the suture 50 in this situation can be attached to an anchor 58 embedded in the cleat material.

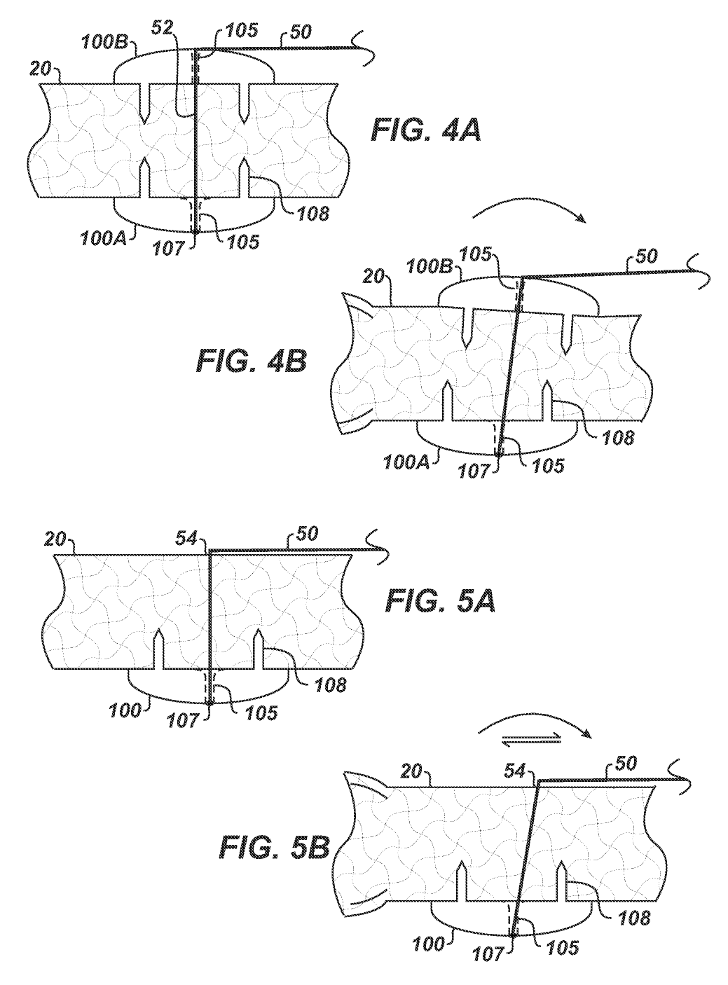

To repair soft tissue injuries, various arrangements of the suture cleats 100 can be used to attach suture to a location in soft tissue that is remote from any distal fixation to bone or the like. In FIG. 4A, for example, two suture cleats 100A-B attach suture 50 at a remote attachment in soft tissue 20 away from distal fixation to bone or other location. As shown, one suture cleat 100A fits on an under side of soft tissue 20 with its spikes 108 embedded therein, while another cleat 100B fits on the upper side of the soft tissue 20 with its spikes 108 also embedded therein. Preferably, the lengths of the spikes 108 are the same on each cleat 100 to provide symmetry in the soft tissue 20 and decrease the stress on the tissue. Yet, these spikes 108 are configured to extend only partially into the soft tissue 20.

One end of suture 50 attaches firmly at 107 to the first cleat 100A's suture passage 105 using one of the various techniques disclosed herein. An intermediate portion 52 of the suture 50 passes from the fixed end at 107, through the soft tissue 20, and through the other cleat 100B's suture passage 105. In this way, the intermediate suture portion 52 stabilizes the two suture cleats 100A-B together while providing a movable connection between them. From the second cleat 100B, the suture 50 can interconnect to another cleat (not shown) at another soft tissue location or can fix distally to bone using a screw, an anchor, a bone tunnel, or the like as disclosed herein. In this way, the suture 50 can act as a tensile member between this attachment location to soft tissue 20 and some other distal attachment.

Because the suture 50 interconnects the cleats 100A-B and acts as the tensile member between them, the suture portion 52's flexible connection prevents the two cleats 10A-B from critically compressing the soft tissue 20, which could produce adverse effects. Furthermore, the flexibility of the suture portion 52 does not constrain the two cleats 100A-B together in one position and can greatly increase their resistance to cyclic loading when compared to a rigid connection. As shown in FIG. 4B, for example, force acting on the suture 50 (due to a change in distance between the attachment locations when soft tissue muscle is flexed or moved) may cause some rotation of the cleats 100A-B. Yet, the flexible connection of the intermediate suture portion 52 may generate less rotation in the cleats 100A-B and more shear force between the cleats 100A-B than would be the case if a rigid connection were instead used.

As shown, the lower cleat 100A firmly attached to the suture 52 experiences less of a moment because the suture 50's force acts closer to this cleat 100A's center of mass. A larger moment is produced on the upper cleat 100B because the suture 50's force acts further from its center of mass. When suture force is applied, the flexibly connected cleats 100A-B may allow the center of the soft tissue 20 between them to remain relatively undisturbed, preventing unnecessary stress concentrations in the area of the greatest bending moment. To prevent substantial disruption of the soft tissue 20 but also to keep the cleats 100A-B embedded, the length of the cleat's spikes 108 can be designed for a particular implementation so that the spikes 108 will not enter the center of the soft tissue 20 and create a stress concentration. Yet, the depth, shape, and location of the spikes 108 on the cleats 100A-B in addition to the width and profile of the cleats 100A-B are preferably selected to prevent the cleats 100A-B from being pulled out. In addition, when the cleats 100A-B tilt, the spikes 108 distribute more of the load from the suture 50 than the surface area of the cleat's body 102. For this reason, several spikes 108 (e.g., three or more) are preferably used on both of the cleats 100A-B. In any event, the arrangement of the cleats 100A-B with interconnecting suture 52 helps to distribute load of the suture 50's force effectively.

Another suture cleat arrangement is shown in FIG. 5A. Here, one cleat 100 fits on one side of the soft tissue 20 with suture 50 firmly attached to the cleat 100 as before, either by tying, engaging, or embedding the suture's end at 107. Because one cleat 100 is used, its spikes 108 may be longer than if two opposing cleats 100 are used. Yet, the spikes 108 preferably do not extend beyond the other side of the soft tissue 20. In contrast to the previous embodiment having two cleats, the suture 50 connected to the cleat 100 passes through the soft tissue 20 and out at a point 54 on the other side, pulling the spikes 108 into and the cleat 100 flush with the soft tissue 20. The other end of the suture 50 then fixes distally as disclosed herein and serves as the tensile member for the distal fixation.

Again, the arrangement of the cleat 100 and suture 50 in FIG. 5A helps to distribute load of the suture 50's force applied to the soft tissue 20 effectively at this attachment location away from the distal fixation to bone or the like. As shown in FIG. 5B, for example, the tissue 20 at the top of the muscle or tendon is compressed only by the suture 50 at point 54. Because a portion of the suture 50's load is not distributed at 54, the shear stress on the tissue 20 may be greater than when two cleats are used (FIG. 4B), but the stress may still be smaller than if no cleats are used on either side of the tissue 20 and only a suture knot were used on the underside of the tissue 20, which could lead to suture pull through. One additional advantage is that the load on the suture 50 actually pulls the cleat 100 toward the tissue 20, preventing the chance of the spikes 108 from pulling out. Moreover, use of the single cleat 100 decreases manufacturing time, associated material cost, and time for surgical implementation.

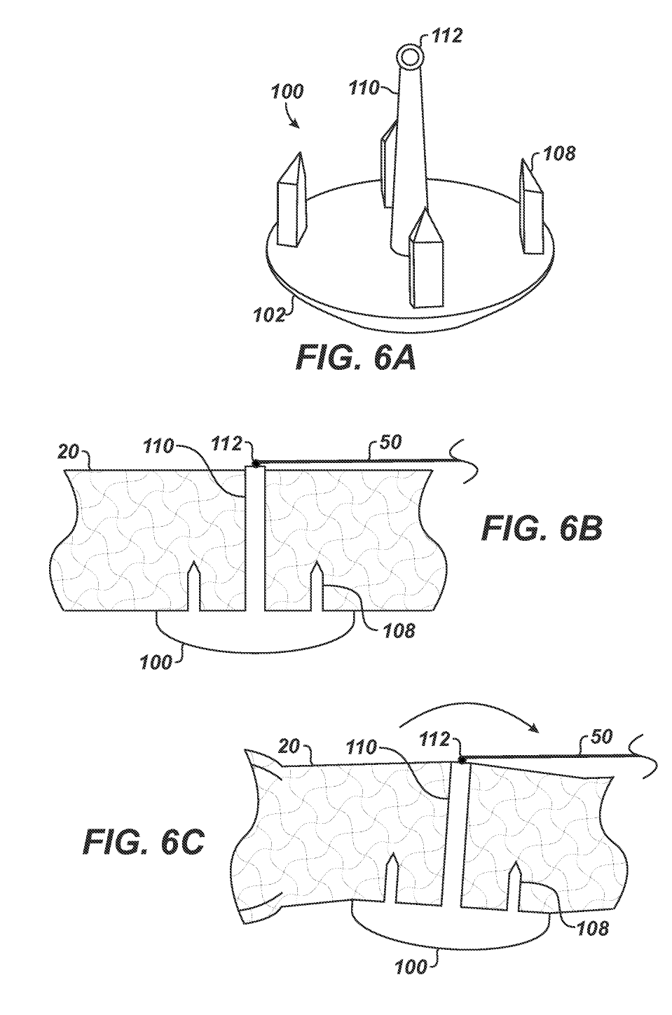

Another suture cleat 100 illustrated in FIG. 6A is similar to previous embodiments and has a disk body 102 and a plurality of spikes 108. In this embodiment, however, the cleat 100 has a post 110 with a distal connection end 112 (e.g., eyelet) for attachment to suture. This post 110 can be rigid or flexible and may be tapered to facilitate positioning the post 110 into soft tissue.

As shown in FIG. 6B, a single one of these cleats 100 fits against one side of the soft tissue 20 with its spikes 108 embedded in the tissue 20 and with its post 110 passing either entirely or partially through the tissue 20. The suture 50 connects to the distal end of the post 110 at the eyelet 112 and exits the soft tissue 20.

As shown in FIG. 6C, the suture cleat 100 with the post 100 may still be subjected to a moment when force is applied by the suture 50. Because the post 110 has a larger diameter than the suture 50, the load of the suture 50's force may be more effectively distributed by the post 100's surface area acting on the adjacent tissue 20. It may be preferable that the post 110 extend slightly through the tissue 20 to the other side enough to extend the thinner suture 50 out of the tissue 20 but not enough for the post 110's end 112 to disturb other tissues, in order that the suture will not put a load on the soft tissue. The length and shape of the post 110 can be designed accordingly for a given implementation. Preferably, the post 110's attachment to the body 102 is strong enough to avoid fatigue failure under cyclical loading.

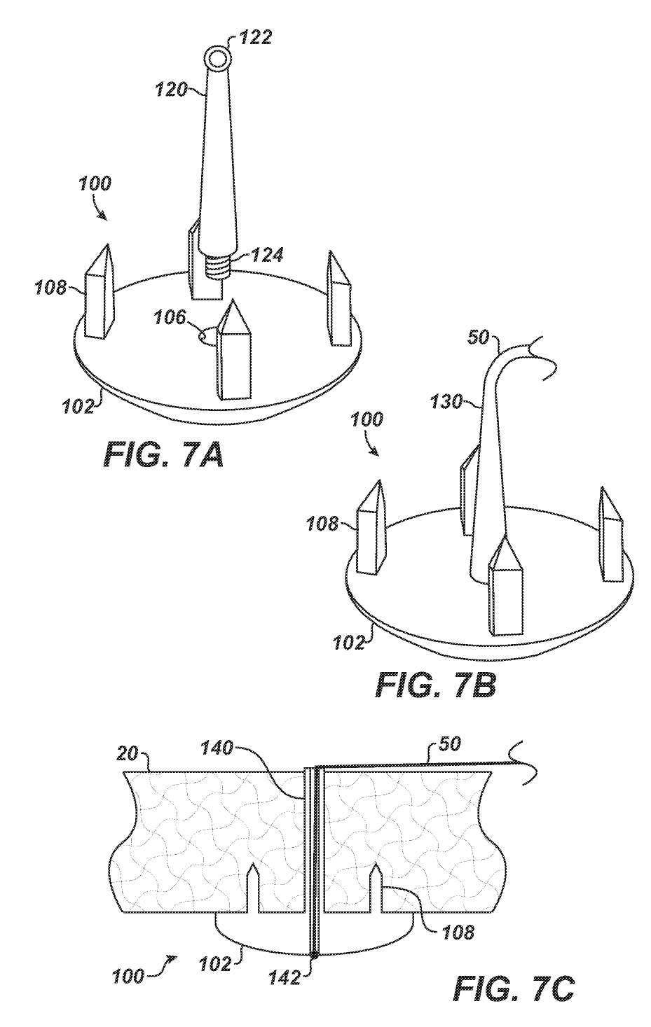

In FIG. 7A, an alternative embodiment of the cleat 100 is shown having an independent post 120 with an eyelet 122 and threaded end 124. As opposed to the integral post 110 of FIG. 6A, the threaded end 124 on this post 120 threads into a threaded opening 106 in the cleat's body 102. In another alternative, the cleat 100 in FIG. 7B has a post 130 that tapers from a thick portion at its connection to the disk body 102. As it tapers, this post 130 forms a thinner, flexible portion that defines an integral length of suture 50 with an end (not shown) that can distally fix to bone or some other device. However, near the exit of the tissue 20, the suture 50 may still be prone to pull through the tissue 20 because the diameter of the suture 50 would return to near its original size. Therefore, it may be preferable that the post 130 extend slightly through the tissue 20 to the other side enough to extend the thinner suture 50 out of the tissue 20 but not enough for the post 110's end 112 to disturb other tissues, in order that the suture will not put a load on the soft tissue 20. The length and shape of the post 130 can be designed accordingly for a given implementation.

In yet another alternative shown in FIG. 7C, the cleat 100 has a post 140 comprised of a hollow tube through which the suture 50 passes. The proximal end of the suture 50 can attach at the base of the post 140 on the other side of the disc body 102 at 142 by a engaging a knot on the end of the suture, by tying the end of the suture 50 to a cross-member, or by one of the other techniques disclosed herein. The suture 50 passes beyond the hollow tube 140 and can then fix distally to bone or some other device.

As disclosed in the above suture cleat arrangements (e.g., FIGS. 4A-5B, 6B-6C, and 7C), the connection of suture 50 to soft tissue by the disclosed cleats 100 is preferably not rigid in nature. This can alleviate a concern associated with connecting suture 50 to soft tissue in a way that overly compresses the soft tissue 20 enough to cause tissue necrosis from lost blood supply, while still connecting suture 50 to soft tissue 20 in a way that is strong enough to prevent pull out of the suture 50 or premature failure. The non-rigid connection of the suture 20 to the soft tissue 20 provided by the suture cleat arrangements can also alleviate concerns associated with a rigid connection such as cyclic loading that could lead to fatigue failure of the connection and require additional surgery to remove free bodies.

Advantageously, the moment generated on the cleats 100 in contact with the soft tissue 20 can provide improved pullout strength. In some cases, the moment is generated on the suture cleat 100 when the muscle contracts. As shown previously in FIGS. 4B, 5B, and 6C, for example, the resulting moment typically causes the suture cleat 100 to tilt with respect to the line of action of the muscle pull, such that portions of the suture cleat 100 are compressed into the soft tissue 20. The tilted cleat 100 has a comparatively large surface area that contacts the soft tissue 20 and advantageously enhances the fixation and pullout strength of the cleat 100 and suture 50.

In the previous discussion, several types of suture cleats 100 have been discussed to which suture 50 attaches for distal fixation to some other mechanism, such as another cleat, a bone tunnel, a screw, or a bone anchor. In the discussion that follows, various arrangements having suture cleats 10 and sutures 50 are described for soft tissue repairs and distal fixation to bone.

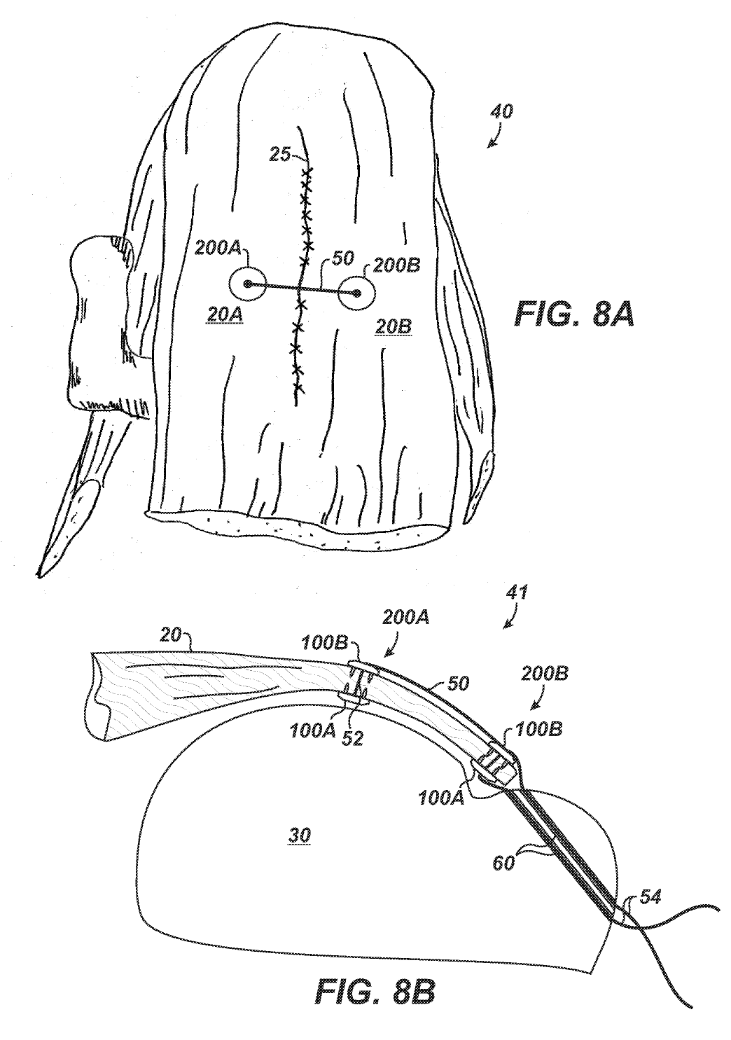

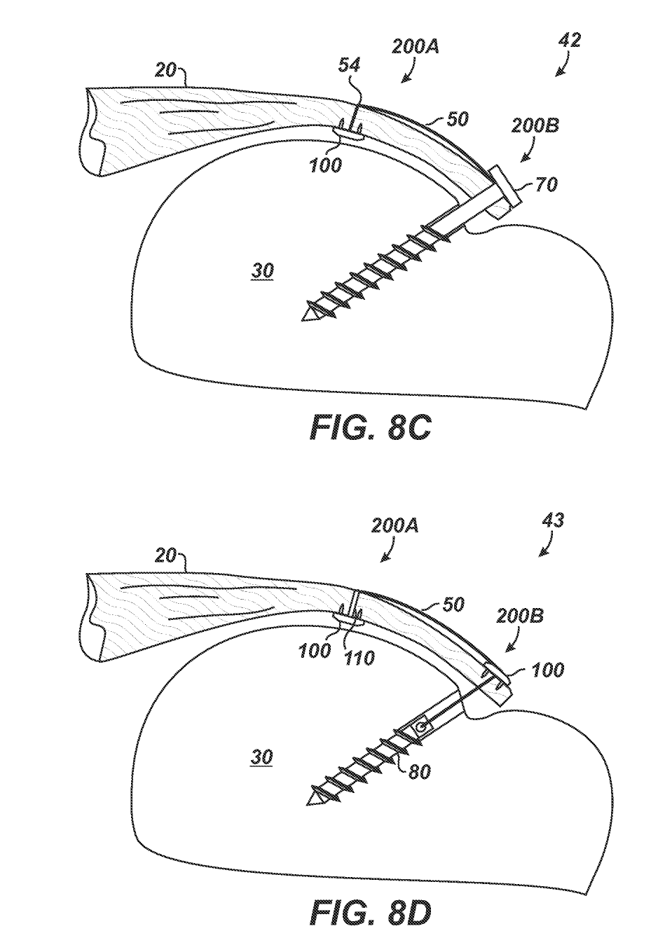

As shown in FIG. 8A, for example, an arrangement 40 of cleat attachments 200A-B and interconnecting suture 50 is used to repair a rotator cuff tear. In this arrangement 40, opposing portions of soft tissue 20A-B are reconnected using cleat attachments 200A-B on both sides of the injury 25. The first cleat attachment 200A connects the interconnecting suture 50 to a healthy portion of the rotator cuff tissue 20A where the tissue 20A is thicker and stronger on one side of the injury 25. At this attachment 200A, a pair of cleats (e.g., 100A-B as in FIG. 4A) can be embedded in the upper and lower surfaces of the soft tissue 20A and interconnected by portion of the suture 50. From the first attachment 200A, the interconnecting suture 50 then spans the torn portion of the rotator cuff to a second attachment 200B on the opposite side of the injury 25 in another portion of healthy soft tissue 20B. Here, a similar pair of cleats (e.g., 100A-B as in FIG. 4A) can be used to fix this end of the suture 50 to the soft tissue 20B. In this way, the cleat attachments 200A-B and suture 50 can augment the soft tissue repair in addition to any standard suturing performed as shown along the injury 25.

In another arrangement 41 shown in FIG. 8B, first and second cleat attachments 200A-B, sutures 50 and 54, and bone tunnels 60 are used to repair soft tissue 20 to bone tissue 30. Here, interconnecting suture 50 attaches to a healthy portion of rotator cuff tissue 20 at the first attachment 200A using a pair of suture cleats 100A-B interconnected by a portion of the suture 52. A length suture 50 then spans from upper cleat 100B and across the tissue. At the second attachment 200B, the suture 50 then reattaches the injured tissue to the healthy bone tissue 30 using an additional pair of suture cleats 100A-B, additional suture 54, and bone tunnels 60.

Typically, in this form of repair, one or more of the bone tunnels 60 are drilled through the bone tissue 30. The suture 54 passes through one tunnel 60, through a portion of the rotator cuff soft tissue 20, through the cleats 100A-B, and through a second tunnel 60 in the bone tissue 30. On the outside of the bone 30, the suture 54 is then tied over a cortical bridge between the tunnels 60. In this way, the cleats 100A-B and sutures 50/54 reattach the soft tissue 20 to the bone 30 in the repair. In an alternative arrangement, one suture cleat 100 can be used at attachment 200B with two sutures 50/54 passing through it and through the bone tunnels 60, 62.

In addition to the use of a bone tunnel, other techniques can be used in conjunction with the disclosed suture cleats 100 of FIGS. 3A through 7C to repair a soft tissue injury to bone tissue. In another arrangement 42 of FIG. 8C, a shoulder soft tissue repair uses one suture cleat 100, a suture 50, and a bone screw 70. In this example, the suture cleat 100 fits against the underside of the soft tissue 20 at attachment 200A and connects one end of suture 50 to a healthy portion of rotator cuff tissue 20. As before, the attachment 200A is proximal to the torn edge where the tissue 20 is thicker and stronger. The suture 50 passes out of the soft tissue 20 at point 54, and the suture 50's other end then connects at attachment 200B directly to the bone screw 70 that reattaches the avulsed tissue 20 to the bone tissue 30.

In yet another arrangement 43 of FIG. 8D, a shoulder soft tissue repair uses suture cleats 100, suture 50, and a suture anchor 80. Here, one end of the suture 50 connects at attachment 200A to soft tissue 20 using a suture cleat 100 having a post 110 as discussed previously. Then, the suture 50 spans the tissue 20 to another suture cleat 100 at attachment 200B. Passing through this cleat 100, the suture 50 passes through a portion of the soft tissue 20 and ties to the suture anchor 80 engaged in the bone tissue 30 of the proximal humerus. The anchor 80 can be a conventional anchor or can be a knotless, friction-type anchor such as the Pushlock Anchor from Arthex Inc. The cleats 100, suture 50, and anchor 80 support the soft tissue 20 through the healing process by facilitating reattachment of the avulsed soft tissue 20 to the bone 30.

In the arrangements 41-43 of FIGS. 8A-8D, attachment to the soft tissue 20 has been shown using pairs of cleats 100A-B (FIG. 8B) on opposing sides of the soft tissue, a single cleat 100 (FIG. 8C) on one side of the soft tissue, and a post-style cleat 100 (FIG. 8D) on one side of the soft tissue. Likewise, attachment to the bone has been accomplished using a pair of suture cleats 100A-B and bone tunnels 60 (FIG. 8B), direct connection to a screw 70 (FIG. 8C), and a single cleat 100 on one side of the tissue and an anchor 80 (FIG. 8C). With the benefit of the present disclosure, however, it will be appreciated that other arrangements and combinations of cleats and bone fixation techniques disclosed herein could also be used.

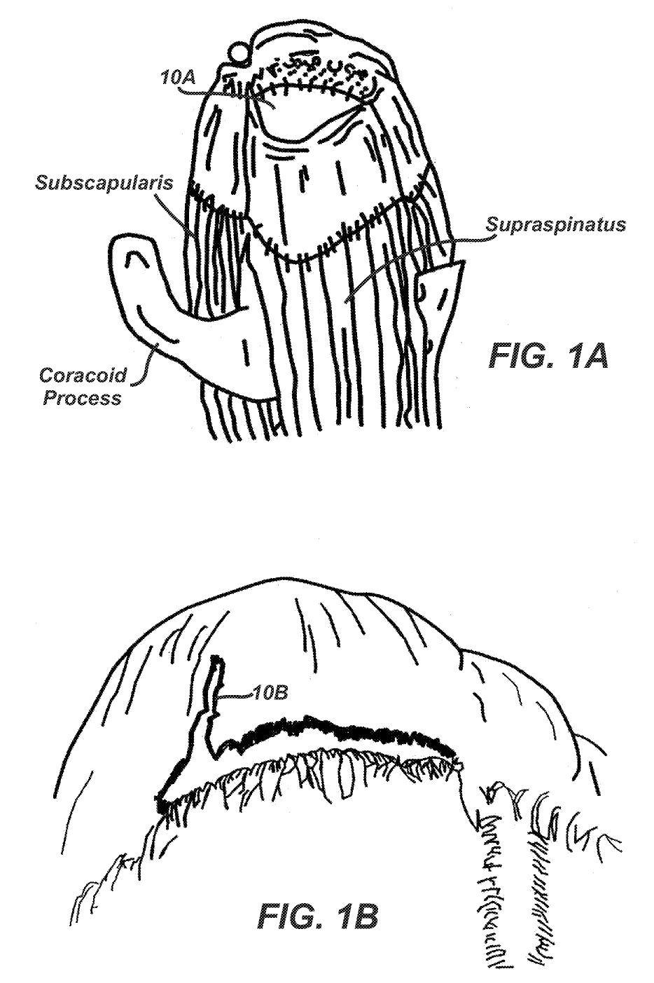

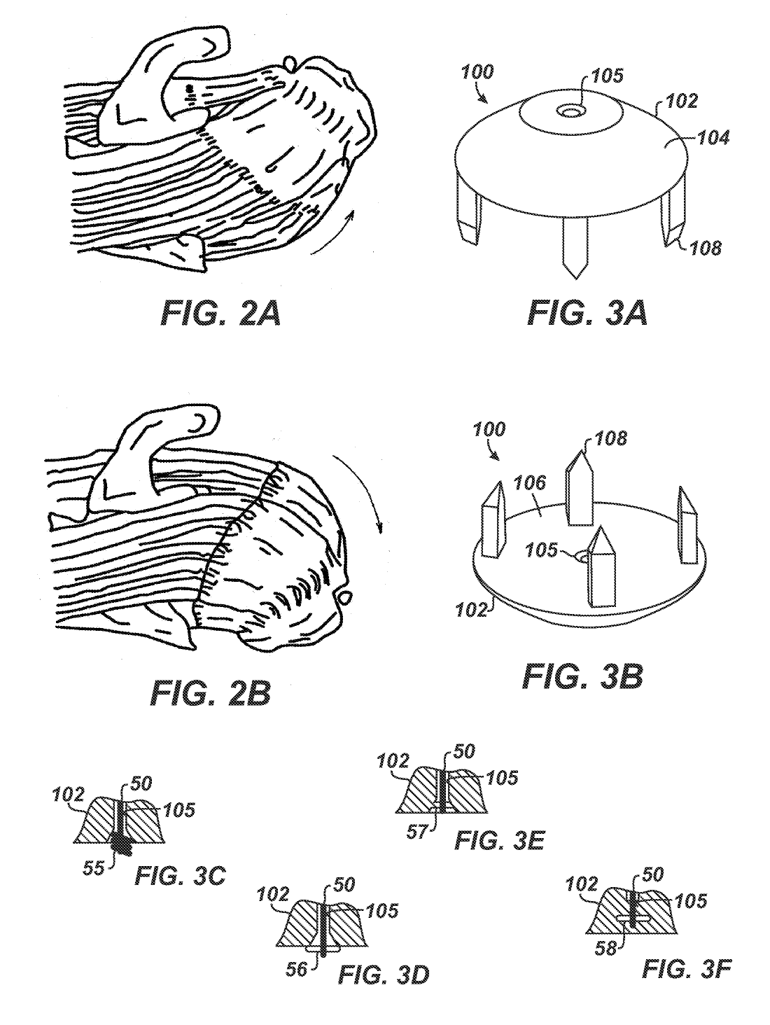

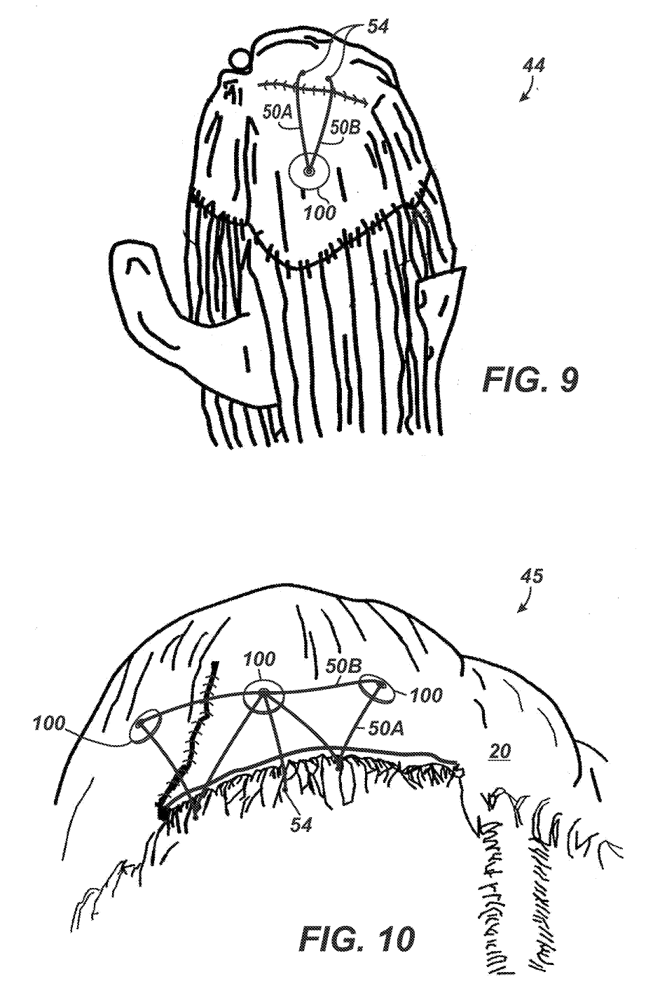

Additional techniques for soft tissue repairs can use a plurality of the disclosed cleats 100 interconnected by various spans of suture 50 as shown in FIGS. 9 and 10. In FIG. 9, a shoulder with a torn rotator cuff is shown repaired using an arrangement 44 of sutures 50A-B and suture cleats 100 of the present disclosure. Here, the tear in the rotator cuff similar to the tear illustrated in FIG. 1A and is repaired using a pair of cleats 100 similar to those disclosed in FIG. 4A. Sutures 50A-B pass from the cleats 100 and attach distally to the bone tissue at points 54 using bone fixation techniques disclosed herein.

In FIG. 10, the shoulder with torn rotator cuff is shown repaired using an arrangement 45 of sutures 50A-B augmented with a plurality of interconnected cleats 100 of the present disclosure. In this case, edges of the torn rotator cuff have been reconnected using sutures 50A that connect from cleats 100 on one side of the injury to points 54 on the other side of the injury, where the suture 50A can connect to another cleat (not shown) on the underside of the tissue 20, to an anchor, etc. In addition, suture 50B interconnects the cleats 100 to each other on the same and different sides of the injury, which may provide even more strength and stability to the repair. In this manner, the present invention may be used to emulate the structure or function of a trestle.

U.S. Pat. Nos. 7,001,411 and 7,303,577 and co-pending application Ser. No. 11/866,220, which are each incorporated herein by reference in their entirety, disclose related soft tissue repair techniques. These related technique use soft tissue cleats that coapt together to attach to soft tissue so that suture can then attach distally to bone. In addition, the related techniques disclosed in U.S. Pat. No. 7,303,577 and co-pending application Ser. No. 11/866,220 use bridge members between attachment locations in repairing soft tissue injuries. By contrast, the repair techniques of the present disclosure do not coapt rigidly on both sides of soft tissue at an attachment location and do not use bridge members between attachment locations. Instead, the present techniques use two suture cleats 100 on both sides of the soft tissue 20 with an interconnecting portion of suture 50 between them (e.g., FIG. 4A); one suture cleat 100 on the underside of soft tissue 20 with suture 50 passing freely through the soft tissue 20 to the tissue's upper side (e.g., FIG. 5A); or one suture cleat 100 on one side of the soft tissue 20 with a post to support the suture 50 on an opposing side of the tissue 20 (e.g., FIG. 6A). In addition, in each of these suture cleat arrangements, suture 50 can pass from these one or more suture cleats 100 at one attachment location to another location where the suture 50 can distally fix to bone or to another cleat arrangement. In this way, the suture 50 provides a tensile interconnection between attachment locations in the present techniques.

As detailed throughout this disclosure, the present techniques for repairing soft tissue provide several benefits beyond what is currently available. As evidenced above, for example, the techniques disclosed herein are intended to limit stress at the attachment location where suture attaches to the soft tissue away from any distal fixation to bone or the like. At this attachment location, the suture cleat arrangements reduce stress to soft tissue at the attachment location and ensure that the attached suture does not pull out when the distance between attachment location changes (e.g., when soft tissue muscle is flexed or stretched).

The foregoing description of preferred and other embodiments is not intended to limit or restrict the scope or applicability of the inventive concepts conceived of by the Applicants. For example, the inventive concepts disclosed herein have been described for use in repair of torn rotator cuffs, and the description and discussion above focus on repairs of rotator cuffs and applications to make such repairs. It will be apparent to those of ordinary skill in the art, however, in light of the present disclosure, that the inventive concepts may apply to other surgical and orthopedic applications. In addition, it will be appreciated that the cleats of the present disclosure may be made of any suitable material for medical purposes, including, but not limited to, a plastic material (e.g., polyethylene, polyetheretherketone, or delrin), a metal material, an elastomeric material, a radiolucent material, a bioabsorbable material, a non-bioabsorbable material, or a combination of these.

In exchange for disclosing the inventive concepts contained herein, the Applicants desire all patent rights afforded by the appended claims. Therefore, it is intended that the appended claims include all modifications and alterations to the full extent that they come within the scope of the following claims or the equivalents thereof.

What is claimed is:

1. A suture attachment apparatus, comprising:
a first body having a first face, the face first positioning against a first side of soft tissue at a first attachment location, the first body supporting a suture such that the suture passes from a second side of the soft tissue at the first attachment location to a second attachment location; and
a plurality of first spikes extending from the first face and engaging the first side,
wherein the apparatus reduces stress to the soft tissue at the first attachment location when a change in distance between the first and second attachment locations occurs.

2. The apparatus of claim 1, wherein the plurality of spikes embed at least partially into the first side of the soft tissue.

3. The apparatus of claim 1, wherein the face distributes force from the suture to an area on the first side of the soft tissue.

4. The apparatus of claim 1, wherein the first body comprises means supporting a proximal end of the suture at the first side of the soft tissue.

5. The apparatus of claim 1, wherein the first body comprises a post extending from the face and into the soft tissue.

6. The apparatus of claim 5, wherein the post comprises means for supporting a proximal end of the suture on a distal end of the post.

7. The apparatus of claim 5, wherein the first body comprises means supporting a proximal end of the suture at the first side of the soft tissue, and wherein the post comprises a passage through which the suture passes.

8. The apparatus of claim 1, further comprising:
a second body having a second face, the second face positioning adjacent the second side of the soft tissue at the first attachment location, the second body supporting the suture such that the suture passes from the first attachment location to the second attachment location; and
a plurality of second spikes extending from the second face and engaging the second side of the soft tissue,
wherein a portion of the suture passing through the soft tissue from the first body to the second body flexibly interconnects the first and second bodies.

9. A suture cleat, comprising:
a body having a face positioning against soft tissue;
a plurality of spikes extending from the face and engaging the soft tissue; and
a post extending from the face and positioning into the soft tissue, the post having a distal end supporting suture.

10. The cleat of claim 9, wherein the distal end of the post comprises means for attaching the suture thereto.

11. The cleat of claim 9, wherein the post comprises a hollow tube through which the suture passes.

12. The cleat of claim 9, wherein the post tapers from a thick portion adjacent the face to a thin portion at the distal end.

13. The cleat of claim 12, wherein the thin portion of the post forms an integral length of the suture having a distally attachable end.

14. A suture system, comprising:
a plurality of suture cleats, each of the suture cleats having a body and a plurality of spikes, the body having a face positioning against soft tissue, the spikes extending from the face and engaging the soft tissue; and
wherein a first of the suture cleats defines a passage therethrough, and
wherein a second of the suture cleats supports a proximal end of suture, the suture passing from the second suture cleat through the soft tissue and passing through the passage in the first suture cleat.

15. The system of claim 14, further comprising a fixation device fixable into bone tissue to which a distal end of the suture attaches.

16. The system of claim 14, wherein the first and second suture cleats position on opposite sides of the soft tissue, and wherein the suture passing through the soft tissue interconnects the first and second suture cleats.

17. The system of claim 14, wherein the second suture cleat comprises means for attaching the proximal end of the suture thereto.

18. The system of claim 14, wherein the second suture cleat comprises a post extending from the face and positioning into the soft tissue.

19. The system of claim 18, wherein the post comprises means for attaching the proximal end of the suture thereto.

20. The system of claim 14, wherein the first suture cleat positions at a first attachment location on a first side of an injury in the soft tissue, and wherein the second suture cleat positions at a second attachment location on a second side of the injury.

21. The system of claim 14, wherein the first and second suture cleats position on a same side of an injury in the soft tissue.

22. A soft tissue injury repair method, comprising:
supporting a proximal end of suture with a first suture cleat;
passing the suture through an under side of soft tissue at a first attachment location;
embedding spikes on the first suture cleat into the underside of the soft tissue;
passing the suture along an upper side of the soft tissue; and
fixing a distal end of the suture at a second attachment location.

23. The method of claim 22, wherein supporting the proximal end of the suture comprises attaching the proximal end to a portion of the first suture cleat.

24. The method of claim 22, further comprising:
passing the suture through a second suture cleat on an upper side of the soft tissue;
embedding spikes on the second suture cleat into the upper side of the soft tissue; and
interconnecting the first and second cleats with portion of the suture.

25. The method of claim 24, wherein embedding the spikes on the second suture cleat comprises embedding the spikes at the first attachment location on the upper side opposite the first suture cleat.

26. The method of claim 24, wherein embedding the spike on the second suture cleat comprises embedding the spikes at the second attachment location away from the first attachment location.

27. The method of claim 22, wherein fixing the distal end of suture comprises fixing the distal end to bone using a fixation device.

28. The method of claim 22, further comprising reducing stress to the soft tissue at the first attachment location when a force is applied to the suture.

29. The method of claim 22, further comprising distributing force from the suture to an area on the under side of the soft tissue with the first suture cleat.
